(19) United States
(12) Patent Application Publication
(10) Pub. No.: US 2019/0328884 A1
(43) Pub. Date: Oct. 31, 2019

(54) COMPOSITIONS AND METHODS OF THEIR USE

(71) Applicant: Chemic Laboratories, Inc., Canton, MA (US)

(72) Inventors: Gerald S. Jones, JR., Norwood, MA (US); Joseph P. St. Laurent, Lakeville, MA (US); Scott A. Goodrich, Stoughton, MA (US)

(21) Appl. No.: 16/005,300

(22) Filed: Jun. 11, 2018

Related U.S. Application Data
(60) Provisional application No. 62/664,772, filed on Apr. 30, 2018.

(51) Int. Cl. A61K 47/34; A61K 36/185; A61K 31/05; A61K 9/00; A61K 47/24

(52) U.S. Cl. CPC A61K 47/34; A61K 36/185; A61K 47/24; A61K 9/0014; A61K 31/05

(57) ABSTRACT

The present invention relates to a composition such as an anhydrous gel, comprising a silicone-based excipient, methods of administering the composition to a subject, and methods of treatment using said compositions.

### CROSS-REFERENCE TO RELATED APPLICATIONS

This application claims priority to U.S. Ser. No. 62/664,772 filed Apr. 30, 2018, the contents of which are incorporated herein by reference.

### BACKGROUND

Traditionally, most active ingredients and pharmaceuticals have been delivered to patients via oral ingestion or injection. Active ingredients or drugs delivered via oral ingestion may take a certain amount of time before they start delivering a therapeutic effect or they may deliver a therapeutic effect for only a short amount of time. Additionally, some people have difficulty ingesting a drug, especially if the drug is included in a relatively large sized pill. Another reason that some oral drug delivery may be problematic is due to high first-pass metabolism. There are a variety of problems associated with injections as well. Most importantly, a majority of people do not enjoy receiving injections. Topically applied formulations avoid a variety of concerns associated with oral and intravenous application methods, including avoidance of first-pass metabolism, possible gastro-intestinal incompatibility and varied conditions of absorption, like pH changes, presence of enzymes, and gastric emptying times. Moreover, topically applied formulations may provide several additional advantages including lower fluctuations in plasma drug levels, ability to more selectively target a specific site for treatment, and ease of treatment. For some conditions, the most effective way to deliver an active is by applying such active directly to the source. Topical formulations that previously described in the prior art still possess several significant limitations such as poor permeability of the drug through the skin from the formulation, low efficiency in delivering the drug by the formulation, residual drugs in the formulation post-application, poor wear characteristics that decrease delivery efficacy and patience compliance, and poor aesthetics that also lead to poor patience compliance. Therefore, there is a need for a new class of topical formulations that improve and overcome the limitations discussed above.

While some topical formulations have been developed in the art to deliver actives to the skin, such formulations have suffered from several important shortcomings. Most significantly, such formulations have been unable to deliver a therapeutic amount of the active ingredient to the skin for an extended period of time. Such formulations tend to deliver a therapeutic amount of the pharmaceutical or healthcare active only for a short period of time, such as for about one or two hours, and the amount of active that is delivered to the skin after one or two hours drops off dramatically, such that little or no therapeutic effect is achieved after about two hours following application to the substrate. Another shortcoming of many formulations known in the art is that they contain water, which requires the use of a significant number of preservatives to prevent or inhibit bacterial growth. Preservatives may be undesirable to some people or in certain applications.

Therefore, there is currently a significant need for a topical formulation that can deliver a therapeutic amount of an active ingredient to the skin for an extended period of time, such as for more than about four, eight or up to 24 hours. Additionally, there is currently a need for a topical formulation that is capable of being free or substantially free of preservatives. Moreover, the active ingredient has to be uniformly incorporated into the topical formulation; in other words, the active ingredient should not include any agglomerates. Finally, the topical formulation should maintain an aesthetic profile and pleasant sensory upon application.

### SUMMARY

The present invention relates to a composition such as an anhydrous gel, and its use for example as a cosmetic base, comprising a silicone-based excipient, e.g., a silicone elastomer, and one or more carrier oils, e.g., one or more natural oils, methods of administering the composition to a subject, e.g., by topical or transdermal administration, and methods of treating disorders comprising administering the composition to the subject.

In one embodiment, the invention comprises a composition comprising a cannabinoid, a silicone elastomer, and silica silylate and is formulated for topical or transdermal administration.

In some embodiments, wherein the composition is anhydrous. In some embodiments, the silicone elastomer is present in the composition in at 25% by weight or less (e.g., 10% by weight or less). In some embodiments, the silicone elastomer is a dimethicone/bis-isobutyl polypropylene glycol crosspolymer or dimethicone/bis-isobutyl PPG-20 crosspolymer. In some embodiments, the composition further comprises isododecane, for example, wherein the weight ratio of isododecane to bis-isobutyl PPG-20 crosspolymer is about 85:15.

In some embodiments, the composition further comprises an oil, for example, the oil is sunflower seed oil, peppermint oil, eucalyptus oil, or cannabis oil. In some embodiments, the composition comprises a plurality of oils. In some embodiments, the composition comprises from about 10 to about 20% by weight of oil (e.g., about 14 to about 16% by weight of oil).

In some embodiments, the composition further comprises a permeation enhance, for example, lauryl lactate, myristyl lactate, cetyl lactate, diethylene glycol monoethyl ether, or methyl laurate.

In some embodiments, the composition comprises an emollient, for example, the emollient can be an ester of a fatty acid such as a triglyceride selected from caprylic/capric triglyceride.

In some embodiments, the composition comprises a silicone fluid such as a trimethicone (e.g., phenyl trimethicone). In some embodiments, the composition further comprises an additional active such as methyl salicylate, camphor, menthol, or a terpene.

In some embodiments, the composition comprises a plurality of cannabinoids. In some embodiments, the cannabinoid is a naturally occurring cannabinoid. In some embodiments, the cannabinoid has been obtained by extraction from a naturally occurring source. In some embodiments, the cannabinoid is synthetic. In some embodiments, the cannabinoid is selected from cannabigerol (CBG), tetrahydrocannabinol (THC), cannabidiol (CBN), cannabichromene (CBC), cannabigerivarin (CBGV), tetrahydrocannabivarian (THCV), cannabidivarin (CBDV), and cannabichromevarin (CBCV).

In one embodiment, the composition is an anhydrous gel. In another embodiment, the anhydrous gel comprises one or more volatile aliphatic hydrocarbons, one or more liquid fatty esters, one or more lactate esters, one or more particular essential oils, and one or more silicone fluids. In another embodiment, the oil is present in the composition at from about 10 to about 20%. In another embodiment, the composition comprises cannabis, a product derived from cannabis, e.g., hemp oil, hemp seed oil, or cannabis oil, or a cannabinoid.

In one aspect, described herein is a composition comprising a silicone-based excipient, a carrier oil, an emollient, and a viscosity enhancement agent, wherein the composition does not comprise a healthcare active. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5%, e.g., about 5%, about 10%, about 20%, or about 30%. In some embodiments, the composition further comprises a permeation enhancer. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1%, e.g., 1%, 3%, 4%, 5%, 10%, 20%, or 30%. In some embodiments, the composition comprises an additional active, e.g., methyl salicylate, camphor, menthol, or a terpene. In some embodiments, the composition further comprises a terpenoid, e.g., camphor. In some embodiments, the composition further comprises a wax, e.g., beeswax. In some embodiments, the composition further comprises cannabis or a product derived from cannabis. e.g., a product derived from cannabis such as hemp oil, hemp seed oil, or cannabis oil. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In one aspect, described herein is a composition comprising a silicone-based excipient, a carrier oil, an emollient, and a viscosity enhancement agent, wherein the composition does not comprise lidocaine, fluocinonide, or tacrolimus. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the oil is present in the composition at from about 10 to about 20%. In some embodiments, the composition further comprises a permeation enhancer. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. e.g., a product derived from cannabis such as hemp oil, hemp seed oil, or cannabis oil. In some embodiments, the composition comprises a cannabinoid, e.g., a cannabinoid described herein. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the cannabinoid is a cannabinoid described herein. In some embodiments, the composition does not comprise diethylene glycol monoethyl ether. In some embodiments, the composition comprises an additional active, e.g., methyl salicylate, camphor, menthol, or a terpene. In some embodiments, the composition further comprises a terpenoid, e.g., camphor. In some embodiments, the composition further comprises a wax, e.g., beeswax. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition formulated for topical or transdermal delivery, the composition comprising a cannabinoid, a carrier oil, a silicone elastomer, and a viscosity enhancement agent, e.g., silica silylate. In some embodiments, the composition is anhydrous, comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the composition comprises a permeation enhancer. In some embodiments, the permeation enhancer is an ester. In some embodiments, the ester is a lactate ester. In some embodiments, the lactate ester is lauryl lactate. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the ester is present in the composition in an amount of less than about 0.5% by weight. In some embodiments, the ester is present in the composition in an amount of from about 0.5% to about 5% by weight. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the cannabinoid is a cannabinoid described herein. In some embodiments, the composition does not comprise diethylene glycol monoethyl ether. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition is substantially free of an antimicrobial. In some embodiments, the composition does not comprise diethylene glycol monoethyl ether. In some embodiments, the composition comprises an additional active, e.g., methyl salicylate, camphor, menthol, or a terpene. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition, the composition comprising a carrier oil, an essential oil, a permeation enhancer, a silicone elastomer, and a cannabinoid. In some embodiments, the composition further comprises a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the permeation enhancer is an ester. In some embodiments, the ester is a lactate ester. In some embodiments, the lactate ester is lauryl lactate. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the composition further comprises an emollient. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the composition does not comprise diethylene glycol monoethyl ether. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. In some embodiments, the cannabinoid is a cannabinoid described herein. In some embodiments, the composition comprises a plurality of cannabinoids, e.g., a plurality of cannabinoids described herein. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In an aspect, described herein is a method of adding an additional component to a composition, e.g., a composition formulated for topical or transdermal administration, comprising a silicone-based excipient, a carrier oil, an emollient, and a viscosity enhancement agent. In some embodiments, the additional component is an essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is added to the composition in an amount that is at least about 1%, e.g., 1%, 3%, 4%, 5%, 10%, 20%, or 30%, of the total weight of the composition. In some embodiments, the additional component comprises cannabis or a product derived from cannabis. In some embodiments, the product derived from cannabis is hemp oil, hemp seed oil, or cannabis oil. In some embodiments, the additional component comprises a cannabinoid, e.g., a cannabinoid described herein. In some embodiments, the additional component comprises a plurality of cannabinoids, e.g., a plurality of cannabinoids described herein. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the additional component comprises an essential oil, e.g., an essential oil described herein, and a cannabinoid, e.g., a cannabinoid described herein. In some embodiments, the additional component comprises an essential oil, e.g., an essential oil described herein, and cannabis or a product derived from cannabis, e.g., a product derived from cannabis such as hemp oil, hemp seed oil, or cannabis oil. In some embodiments the cannabinoid is derived from a naturally-occurring source, e.g., a natural oil such as hemp oil, hemp seed oil, or cannabis oil. In some embodiments, the essential oil is present in the composition in at least about 1% by weight, e.g., 1%, 3%, 4%, 5%, 10%, 20%, or 30% by weight. In some embodiments, the cannabinoid is present in the composition in at least about 1% by weight, e.g., 1%, 3%, 4%, 5%, 10%, 20%, or 30% by weight. In some embodiments, the additional component comprises a terpenoid, e.g., camphor. In some embodiments, the additional component comprises an additional active, e.g., methyl salicylate, camphor, menthol, or a terpene. In some embodiments, the composition is administered on a keratinous surface of the subject. In some embodiments, the keratinous surface is the skin of the subject. In some embodiments, the subject is a mammal. In some embodiments, the mammal is a human.

In an aspect, described herein is a method of treating a disorder in a subject in need thereof, comprising administering to the subject a composition described herein. In some embodiments, the disorder is selected from the group consisting of nausea, pain, epilepsy, refractory spasticity, anorexia, neuropathic pain, inflammation, Crohns disease, gout, opioid dependence, insomnia, psoriasis, shingles, rheumatoid arthritis, migraine, Dravet syndrome, glaucoma, and cancer.

### DETAILED DESCRIPTION

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 carbon atoms in it. The term “haloalkyl” refers to an alkyl in which one or more hydrogen atoms are replaced by halo, and includes alkyl moieties in which all hydrogens have been replaced by halo, e.g., perfluoroalkyl. The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “aryl” refers to an aromatic monocyclic, bicyclic, or tricyclic hydrocarbon ring system, wherein any ring atom capable of substitution can be substituted, e.g., by one or more substituents. Examples of aryl moieties include, but are not limited to, phenyl, naphthyl, and anthracenyl.

The term “arylalkyl” or the term “aralkyl” refers to alkyl substituted with an aryl. Exemplary aralkyls include but are not limited to benzyl and phenethyl.

The term “cycloalkyl” as employed herein includes saturated cyclic, bicyclic, tricyclic, or polycyclic hydrocarbon groups having 3 to 12 carbons. Any ring atom can be substituted, e.g., by one or more substituents. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100=80%.

Thus, a composition containing 90% of one enantiomer and 10% of the other enantiomer is said to have an enantiomeric excess of 80%. Some of the compositions described herein contain an enantiomeric excess of at least 50%, 75%, 90%, 95%, or 99% of Compound 1 (the S-enantiomer). In other words the compositions contain an enantiomeric excess of the S enantiomer over the R enantiomer.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. The heteroatom may optionally be the point of attachment of the heterocyclyl substituent. Any ring atom can be substituted, e.g., by one or more substituents. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. Any ring atom can be substituted, e.g., by one or more substituents.

As used herein, the term “about” denotes an approximate range of plus or minus 10% from a specified value. For instance, the language “about 20%” encompasses a range of 18-22%. As used herein, “about” also includes the exact amount. Hence “about 20%” means “about 20%” and also “20%.”

“Anhydrous,” as used herein, means a water content of less than or equal to 3% by weight, for example, less than 2% by weight or less than 1% by weight, relative to the weight of the composition, including 0% water. If present, water in the composition may be “bound water,” for instance the water of crystallization of salts, or traces of water absorbed by the starting materials used in the preparation of the anhydrous compositions according to the present disclosure.

A “keratinous substrate”, as used herein, includes but is not limited to, skin, hair, eyelashes, lips and nails.

As used herein, “emollient” is a hydrophobic material that provides softness, lubricity and smoothness to the skin and often forms a thin occlusive film which increases hydration by reducing transepidermal water loss (TEWL).

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

The term “substantially free” when referring to a compound or composition described herein means that there is less than 20% (by weight) of the designated compound or by-product (e.g., a saturated alcohol starting material) present, more preferably, there is less than 10% (by weight) of the designated compound or by-product, more preferably, there is less than 9% (by weight) of the designated compound or by-product, more preferably, there is less than 8% (by weight) of the designated compound or by-product, more preferably, there is less than 7% (by weight) of the designated compound or by-product, more preferably, there is less than 6% (by weight) of the designated compound or by-product, more preferably, there is less than 5% (by weight) of the designated compound or by-product, more preferably, there is less than 4% (by weight) of the designated compound or by-product, more preferably, there is less than 3% (by weight) of the designated compound or by-product, more preferably, there is less than 2% (by weight) of the designated compound or by-product, and most preferably, there is less than 1% (by weight) of the designated compound or by-product.

The term “substituents” refers to a group “attached” to a alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, e.g., C1, C2, C3, C4, C5, C6, C7, C8, C9, C10, C11, C12 straight or branched chain alkyl, cycloalkyl, haloalkyl, e.g., perfluoroalkyl such as CF₃, aryl, heteroaryl, aralkyl, heteroaralkyl, heterocyclyl, alkenyl, alkynyl, cycloalkenyl, heterocycloalkenyl, alkoxy, haloalkoxy, e.g., perfluoroalkoxy such as OCF₃, halo, hydroxy, carboxy, carboxylate, cyano, nitro, amino, alkyl amino, SO₃H, sulfate, phosphate, methylenedioxy e.g., —O—CH₂—O—, ethylenedioxy, oxo, thioxo, e.g., C=S, imino, e.g., alkyl, aryl, aralkyl, S(O)ₙalkyl, S(O)ₙaryl, S(O)ₙheteroaryl, S(O)ₙheterocyclyl, i.e., wherein is an integer between 0 and 2, amine, e.g., mono-, di-, alkyl, cycloalkyl, aralkyl, heteroaralkyl, aryl, heteroaryl, and combinations thereof, ester, e.g., alkyl, aralkyl, heteroaralkyl, aryl, heteroaryl, amide, e.g., mono-, di-, alkyl, aralkyl, heteroaralkyl, aryl, heteroaryl, and combinations thereof, sulfonamide, e.g., mono-, di-, alkyl, aralkyl, heteroaralkyl, and combinations thereof. In one aspect, the substituents on a group are independently any one single, or any subset of the aforementioned substituents. In another aspect, a substituent may itself be substituted with any one of the above substituents.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human” and “subject” are used interchangeably herein.

As used herein, “anhydrous” means that, other than water of hydration contained in the various components used to formulate the product, no free water is added to the composition. Typically, the water content of an anhydrous composition described herein will be less than 5% by weight. Preferably the water content of the composition is less than 3% and most preferably less than about 1% by weight of the composition.

As used herein, the term “permeation enhancer” refers to an agent or mixture of agents which acts to increase the permeability of the skin to therapeutic agents.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”).

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### Compositions

The present invention relates to a composition such as an anhydrous gel, and its use for example as a cosmetic base, comprising a silicone-based excipient, e.g., a silicone elastomer, and one or more carrier oils, e.g., one or more natural oils, methods of administering the composition to a subject, e.g., by topical or transdermal administration, and methods of treating disorders comprising administering the composition to the subject.

It has now been discovered that, by the judicious combination of a silicone elastomer and one or more natural oils, in an anhydrous composition based on volatile aliphatic hydrocarbon(s), liquid fatty esters, lactate esters, particular essential oils and silicone fluids, it is possible to realize clear, gel systems with enhanced aesthetics that impart a luxurious, silky, non-greasy skin feel. Moreover these anhydrous compositions are compatible with a wide range of excipients and other organic ingredients, and perform as quick-absorbing delivery systems for lipophilic actives. In addition, these compositions are water repellant, skin protectant, and amenable to cold processing.

Silicone elastomers are used in the cosmetic industry as thickening agents for both water-based and anhydrous formulations and to enhance the aesthetics of those formulations and provide a novel form of delivery for other formulation components. The elastomers are, in general, compatible with a wide range of organic compounds, including natural oils and silicone fluids. Natural oils are used in cosmetic formulations to provide emolliency and to help maintain the skin barrier and decrease transepidermal water loss (TEWL). For example, sunflower oil, which is rich in linoleic acid, may be particularly beneficial in these respects. Unavoidable consequences of an oil-rich formulation can be an undesirable greasy feel, and long-term residence on the skin. It has been found that the greasy feel of an oil-rich formulation can be mitigated by admixture with silicone elastomer and silicone fluids.

The anhydrous compositions of the instant invention can be rich in emollients like liquid fatty esters and triglycerides and contain a variety of other functional excipients like lactate esters as penetration enhancers; aliphatic hydrocarbons as diluents and to enhance spreadability as well as to help prevent TEWL; silicone fluids to decrease tackiness and enhance overall aesthetics of formulations and for recognized skin protectant effects; and silica silylate for high efficiency viscosity enhancement and fragrance retention. In one embodiment, the compositions comprise cannabis, a product derived from cannabis, e.g., hemp oil, hemp seed oil, or cannabis oil, or a cannabinoid.

In one aspect, described herein is a composition comprising a silicone-based excipient, a carrier oil, an emollient, and a viscosity enhancement agent, wherein the composition does not comprise a healthcare active. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5%, e.g., about 5%, about 10%, about 20%, or about 30%. In some embodiments, the composition further comprises a permeation enhancer. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1%, e.g., 1%, 3%, 4%, 5%, 10%, 20%, or 30%. In some embodiments, the composition comprises an additional active, e.g., methyl salicylate, camphor, menthol, or a terpene. In some embodiments, the composition further comprises a terpenoid, e.g., camphor. In some embodiments, the composition further comprises a wax, e.g., beeswax. In some embodiments, the composition further comprises cannabis or a product derived from cannabis. e.g., a product derived from cannabis such as hemp oil, hemp seed oil, or cannabis oil. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In one aspect, described herein is a composition comprising a silicone-based excipient, a carrier oil, an emollient, and a viscosity enhancement agent, wherein the composition does not comprise lidocaine, fluocinonide, or tacrolimus. In some embodiments, the viscosity enhancement agent is silica siliylate. In some embodiments, the silicone-based excipient is present in the composition in at least about 80% by weight. In some embodiments, the a silicone-based excipient is a silicone elastomer. In some embodiments, the silicone elastomer is provided in a carrier fluid. In some embodiments, the silicone elastomer is dimethicone/bis-isobutyl polypropylene glycol crosspolymer, e.g., dimethicone/bis-isobutyl PPG-20 crosspolymer. In some embodiments, the carrier fluid is isododecane. In some embodiments, the weight ratio of carrier fluid to silicone elastomer is about 85:15. In some embodiments, the carrier oil is a natural oil. In some embodiments, the carrier oil is selected from the group consisting of sunflower seed oil, coconut oil, grape seed oil, olive oil, jojoba, and combinations thereof. In some embodiments, the carrier oil is sunflower seed oil. In some embodiments, the carrier oil is present in the composition in at least about 5% by weight, e.g., about 5%, about 10%, about 20%, or about 30% by weight. In some embodiments, the composition further comprises a permeation enhancer. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition further comprises a silicone fluid. In some embodiments, the silicone fluid is a trimethicone. In some embodiments, the trimethicone is phenyl trimethicone. In some embodiments, the composition is an anhydrous gel. In some embodiments, the composition comprises less than 0.5% of water by weight. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. e.g., a product derived from cannabis such as hemp oil, hemp seed oil, or cannabis oil. In some embodiments, the composition comprises a cannabinoid, e.g., a cannabinoid described herein. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the cannabinoid is a cannabinoid described herein. In some embodiments, the composition does not comprise diethylene glycol monoethyl ether. In some embodiments, the composition comprises an additional active, e.g., methyl salicylate, camphor, menthol, or a terpene. In some embodiments, the composition further comprises a terpenoid, e.g., camphor. In some embodiments, the composition further comprises a wax, e.g., beeswax. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition formulated for topical or transdermal delivery, the composition comprising a cannabinoid, a carrier oil, a silicone elastomer, and a viscosity enhancement agent, e.g., silica silylate. In some embodiments, the composition is anhydrous, comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the composition comprises a permeation enhancer. In some embodiments, the permeation enhancer is an ester. In some embodiments, the ester is a lactate ester. In some embodiments, the lactate ester is lauryl lactate. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the ester is present in the composition in an amount of less than about 0.5% by weight. In some embodiments, the ester is present in the composition in an amount of from about 0.5% to about 5% by weight. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition comprises a plurality of cannabinoids. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the cannabinoid is a cannabinoid described herein. In some embodiments, the composition does not comprise diethylene glycol monoethyl ether. In some embodiments, the composition further comprises an additional essential oil. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition is substantially free of an antimicrobial. In some embodiments, the composition does not comprise diethylene glycol monoethyl ether. In some embodiments, the composition comprises an additional active, e.g., methyl salicylate, camphor, menthol, or a terpene. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In another aspect, described herein is a composition, the composition comprising a carrier oil, an essential oil, a permeation enhancer, a silicone elastomer, and a cannabinoid. In some embodiments, the composition further comprises a viscosity enhancement agent, e.g., silica silylate. In some embodiments, the composition is anhydrous, e.g., comprising less than 0.5% of water by weight. In some embodiments, the composition is an anhydrous gel. In some embodiments, the silicone-based excipient is present in the composition in at least about 80% by weight. In some embodiments, the silicone elastomer is dimethicone/bis-isobutyl polypropylene glycol crosspolymer, e.g., dimethicone/bis-isobutyl PPG-20 crosspolymer. In some embodiments, the silicone elastomer is provided in a carrier fluid. In some embodiments, the carrier fluid is isododecane. In some embodiments, the weight ratio of carrier fluid to silicone elastomer is about 85:15. In some embodiments, the composition further comprises an oil, e.g., a natural oil, e.g., sunflower seed oil, peppermint oil, eucalyptus oil, cannabis oil, or a combination thereof. In some embodiments, the permeation enhancer is an ester. In some embodiments, the ester is a lactate ester. In some embodiments, the lactate ester is lauryl lactate. In some embodiments, the permeation enhancer is selected from the group consisting of lauryl lactate, myristyl lactate, cetyl lactate, diethylene glycol monoethyl ether, and combinations thereof. In some embodiments, the permeation enhancer comprises one or more esters described herein. In some embodiments, the permeation enhancer consists essentially of one or more esters described herein. In some embodiments, the permeation enhancer consists of one or more esters described herein. In some embodiments, the composition further comprises an emollient. In some embodiments, the emollient comprises one or more esters described herein. In some embodiments, the emollient consists essentially of one or more esters described herein. In some embodiments, the emollient consists of one or more esters described herein. In some embodiments, the emollient is an ester of a fatty acid, e.g., a triglyceride. In some embodiments, the emollient is caprylic/capric triglyceride. In some embodiments, the emollient comprises a caprylic triglyceride. In some embodiments, the emollient comprises a capric triglyceride. In some embodiments, the emollient is selected from the group consisting of lauryl lactate, myristyl lactate, cetyl lactate, diethylene glycol monoethyl ether, and combinations thereof. In some embodiments, the composition is substantially free of an antimicrobial. In some embodiments, the composition further comprises a silicone fluid. In some embodiments, the silicone fluid comprises a trimethicone. In some embodiments, the trimethicone is phenyl trimethicone. In some embodiments, the composition does not comprise diethylene glycol monoethyl ether. In some embodiments, the essential oil is selected from the group consisting of birch oil, lavender oil, eucalyptus oil, peppermint oil, a high terpene oil, and combinations thereof. In some embodiments, the essential oil is selected from the group consisting of lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is present in the composition in at least about 1% by weight, e.g., 1%, 3%, 4%, 5%, 10%, 20%, or 30% by weight. In some embodiments, the composition further comprises cannabis or a product derived from cannabis. In some embodiments, the cannabinoid is a cannabinoid described herein. In some embodiments, the composition comprises a plurality of cannabinoids, e.g., a plurality of cannabinoids described herein. In some embodiments, the cannabinoid is derived from a naturally occurring source, e.g., cannabis oil, hemp oil, and hemp seed oil. In some embodiments, the cannabinoid is a synthetic cannabinoid. In some embodiments, the composition is substantially free of an antimicrobial. In some embodiments, the composition does not comprise diethylene glycol monoethyl ether. In some embodiments, the composition is formulated for topical administration. In some embodiments, the composition is administered on a keratinous surface of the subject. In some embodiments, the keratinous surface is the skin of the subject. In some embodiments, the subject is a mammal. In some embodiments, the mammal is a human.

In an aspect, described herein is a method of adding an additional component to a composition, e.g., a composition formulated for topical or transdermal administration, comprising a silicone-based excipient, a carrier oil, an emollient, and a viscosity enhancement agent. In some embodiments, the additional component is an essential oil. In some embodiments, the essential oil is selected from the group consisting of birch oil, lavender oil, eucalyptus oil, peppermint oil, a high terpene oil, and combinations thereof. In some embodiments, the high terpene oil comprises myrcene, linalool, limonene, pinene, eucalyptol, humalene, borneol, terpinolene, or combinations thereof. In some embodiments, the essential oil is added to the composition in an amount that is at least about 1%, e.g., 1%, 3%, 4%, 5%, 10%, 20%, or 30%, of the total weight of the composition. In some embodiments, the additional component comprises cannabis or a product derived from cannabis. In some embodiments, the product derived from cannabis is hemp oil, hemp seed oil, or cannabis oil. In some embodiments, the additional component comprises a cannabinoid, e.g., a cannabinoid described herein. In some embodiments, the additional component comprises a plurality of cannabinoids, e.g., a plurality of cannabinoids described herein. In some embodiments, the plurality of cannabinoids comprises cannabidiol and tetrahydrocannabinol. In some embodiments, the cannabidiol comprises a synthetic cannabidiol. In some embodiments, the cannabidiol is derived from a natural oil, e.g., cannabis oil, hemp oil, or hemp seed oil. In some embodiments, the additional component comprises an essential oil, e.g., an essential oil described herein, and a cannabinoid, e.g., a cannabinoid described herein. In some embodiments, the additional component comprises an essential oil, e.g., an essential oil described herein, and cannabis or a product derived from cannabis, e.g., a product derived from cannabis such as hemp oil, hemp seed oil, or cannabis oil. In some embodiments the cannabinoid is derived from a naturally-occurring source, e.g., a natural oil such as hemp oil, hemp seed oil, or cannabis oil. In some embodiments, the essential oil is present in the composition in at least about 1% by weight, e.g., 1%, 3%, 4%, 5%, 10%, 20%, or 30% by weight. In some embodiments, the cannabinoid is present in the composition in at least about 1% by weight, e.g., 1%, 3%, 4%, 5%, 10%, 20%, or 30% by weight. In some embodiments, the additional component comprises a terpenoid, e.g., camphor. In some embodiments, the additional component comprises an additional active, e.g., methyl salicylate, camphor, menthol, or a terpene. In some embodiments, the composition is administered on a keratinous surface of the subject. In some embodiments, the keratinous surface is the skin of the subject. In some embodiments, the subject is a mammal. In some embodiments, the mammal is a human.

The compositions described herein can be formulated for topical or transdermal administration. In an embodiment, a compositions described herein is topically administered to a keratinous substrate on the subject, e.g., the skin of a human.

Transdermal compositions are typically formulated as a topical ointment, lotion, cream, or anhydrous gel containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compositions provided herein can also be administered by a transdermal device. Accordingly, transdermal administration can be accomplished using a patch either of the reservoir or porous membrane type, or of a solid matrix variety.

### Carrier Oils

The compositions of the present invention can comprise a carrier oil, e.g., a natural oils, e.g., oils derived from plants. Non-limiting examples of natural oils include sunflower seed oil, coconut oil, grape seed oil, hemp seed oil, olive oil, hemp oil, and jojoba. The composition can comprise a combination of carrier oils, including but not limited to any combination of sunflower seed oil, coconut oil, grape seed oil, hemp seed oil, olive oil, hemp oil, and jojoba.

In some embodiments, the carrier oil is present in the composition from about 1% by weight to about 90% by weight. In some embodiments, the carrier oil is present in the composition from about 1% by weight to about 50% by weight. In some embodiments, the carrier oil is present in the composition from about 1% by weight to about 40% by weight. In some embodiments, the carrier oil is present in the composition from about 1% by weight to about 30% by weight. In some embodiments, the carrier oil is present in the composition from about 1% by weight to about 20% by weight. In some embodiments, the carrier oil is present in the composition from about 1% by weight to about 10% by weight. In some embodiments, the carrier oil is present in the composition from about 1% by weight to about 5% by weight. In some embodiments, the carrier oil is present in the composition in about 30% by weight, e.g., 30% by weight. In some embodiments, the carrier oil is present in the composition in about 20% by weight, e.g., 20% by weight. In some embodiments, the carrier oil is present in the composition in about 15% by weight, e.g., 15% by weight. In some embodiments, the carrier oil is present in the composition in about 14.5% by weight, e.g., 14.5% by weight. In some embodiments, the carrier oil is present in the composition in about 14% by weight, e.g., 14% by weight. In some embodiments, the carrier oil is present in the composition in about 13.7% by weight, e.g., 13.7% by weight. In some embodiments, the carrier oil is present in the composition in about 10% by weight, e.g., 10% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Silicone-Based Excipient

The silicone-based excipient may be any silicone-containing polymer material, including a silicone elastomer blend, a silicone organic elastomer blend, a silicone resin, a silicone elastomer, a pressure sensitive adhesive, a silicone gum, a silicone wax, an elastomer base sealant, adhesive or any combination thereof. The silicone-based excipient may be a dimethicone cross polymer, a dimethicone/bis-isobutyl propylene glycol cross polymer, a polyethylene glycol-12 dimethicone/bis-isobutyl propylene glycol-20 cross polymer, or any combination thereof.

Silicones are a class of compounds based on polydialkylsiloxanes. Silicones have been used extensively to enhance aesthetics of personal care formulations by providing a unique sensory profile upon application. Silicone elastomer gels are generally obtained by a crosslinking hydrosilylation reaction of a SiH polysiloxane with another polysiloxane containing an unsaturated hydrocarbon substituent, such as a vinyl functional polysiloxane, or by crosslinking a SiH polysiloxane with a hydrocarbon diene. The silicone elastomers may be formed in the presence of a carrier fluid, such as a volatile silicone, resulting in a gelled formulation. In some embodiments, the carrier fluid is isododecane.

The silicone-based excipient may be a pressure sensitive adhesive (PSA). The PSA may be the reaction product of a hydroxyl end-blocked polydimethylsiloxane polymer and a hydroxy functional silicate resin. The polymer and resin react in a condensation reaction to form the PSA. The advantage of using the PSA as the silicone component is the substantivity that the PSA provides. The substantivity is particularly advantageous in human and veterinary applications that require significant substantivity for the active agent to provide sustained pharmacological effects.

For purposes of the present disclosure, the terms “silicone rubber” and “silicone elastomer” are synonymous, at least to the extent that both silicone components are capable of elongation and recovery. The silicone elastomers may be contained in a carrier fluid such as cyclopentasiloxane, isododecane, isodecylneopentanoate, caprylyl methicone, or other suitable carrier fluids. Silicone rubbers and silicone elastomers are generally crosslinked or reacted silicone polymers. In contrast, silicone gums are capable of being stretched, but they do not generally snap back. Silicone gums are the high molecular weight, generally linear, polydiorganosiloxanes that can be converted from their highly viscous plastic state into a predominately elastic state by crosslinking. Silicone gums are often used as one of the main components in the preparation of silicone rubbers and silicone elastomers.

The compositions described herein can comprise one or more silicone elastomers. In some embodiments, the silicone elastomer is a dimethicone/bis-isobutyl polypropylene glycol crosspolymer, e.g., dimethicone/bis-isobutyl PPG-20 cross polymer. In some embodiments, the ratio of dimethicone to bis-isobutyl polypropylene glycol in the cross polymer is 85:15.

The silicone resins may include MQ resins. The acronym MQ as it relates to silicone resins is derived from the symbols M, D, T, and Q each of which represent a functionality of different types of structural units which may be present in silicone resins containing siloxane units joined by Si—O—Si bonds. Monofunctional (M) unit represents (CH₃)₃SiO_{1/2}. Difunctional (D) unit represents (CH₃)₂SiO_{2/2}. Trifunctional (T) unit represents CH₃SiO_{3/2} and results in the formation of branched linear siloxanes. Tetrafunctional (Q) unit represents SiO_{4/2} which results in the formation of crosslinked and resinous silicone compositions. Hence, MQ is used when the siloxane contains all monofunctional M and tetrafunctional Q units, or at least a high percentage of M and Q units such as to render the silicone resinous.

Silicone resins may include non-linear siloxane resins having a glass transition temperature (T_{g}) above about 0° C. Glass transition temperature is the temperature at which an amorphous material such as a higher silicone polymer changes from a brittle vitreous state to a plastic state. The silicone resin generally has the formula R′ₐSiO_{(4-a)/2} wherein R′ is a monovalent hydrocarbon group with 1-6 carbon atoms or a functionally substituted hydrocarbon group with 1-6 carbon atoms, and a has an average value of 1-1.8. The silicone resin will preferably include monofunctional (M) units R″₃SiO_{1/2} and tetrafunctional (Q) units SiO_{4/2}, in which R″ is the monovalent hydrocarbon group having 1-6 carbon atoms, most preferably the methyl group. The number ratio of M groups to Q groups may be in the range of 0.5:1 to 1.2:1, so as to provide an equivalent wherein a in the formula R′ₐSiO_{(4-a)/2} has an average value of 1.0-1.63. The number ratio of M groups to Q groups may also be between about 0.6:1 to about 0.9:1. Silicone MQ resins in which the number of Q units per molecule is higher than 1 or higher than 5 may also be used.

The silicone resin may also contain between about 1 to about 5% by weight of silicon-bonded hydroxyl radicals such as a dimethylhydroxysiloxy unit (HO)(CH₃)₂SiO_{1/2}. If desired, the silicone resin may contain minor amounts of difunctional (D) units and/or trifunctional (T) units. Silicone resins having a viscosity of at least 100,000,000 (100 million) centistoke (mmf²/s) and a softening temperature of less than about 200° C. may also be used. The silicone resin may include (i) silicone resins of the type Mₓ% where x and y have values such that the silicone resin contains at least more than 5 Q units per molecule; (ii) silicone resins of the type MₓT_{y} where x and y have values such that the silicone resin contains at least more than 5 T units per molecule; and (iii) silicone resins of the type MₓD_{y}TₚQ_{q} where x, y, p, and q have values such that the sum of Q and T units is at least more than 5 units per molecule, and the number of D units varies from 0-100.

The compositions described herein can comprise one or more silicone-based excipients. In some embodiments, the silicone-based excipient, e.g., a silicone elastomer provided in a carrier fluid such as isododecane, is present in the composition in least about 50% by weight, in at least about 55% by weight, in least about 60% by weight, in at least about 65% by weight, in least about 70% by weight, in at least about 75% by weight, in at least about 80% by weight, in at least about 85% by weight, in at least about 90% by weight, in at least about 95% by weight, or in at least about 99% by weight. In some embodiments, the silicone-based excipient, e.g., a silicone elastomer provided in a carrier fluid such as isododecane, is present in the composition in least about 55% by weight, e.g., 55% by weight. In some embodiments, the silicone-based excipient, e.g., a silicone elastomer provided in a carrier fluid such as isododecane, is present in the composition in least about 55% by weight, e.g., 55% by weight. In some embodiments, the silicone-based excipient, e.g., a silicone elastomer provided in a carrier fluid such as isododecane, is present in the composition in least about 65% by weight, e.g., 65% by weight. In some embodiments, the silicone-based excipient, e.g., a silicone elastomer provided in a carrier fluid such as isododecane, is present in the composition in least about 67.4% by weight, e.g., 67.4% by weight.

### Other Excipients

In addition to active agent and silicone-based excipients, various excipients may be incorporated into the topical formulation. As generally understood by those skilled in the art, excipients are additives that are used to convert the active agent into appropriate dosage forms that are suitable for application to the substrate. Excipients may also be added to stabilize the formulation and to optimize application characteristics, such as flowability.

Examples of potential excipients include, but are not limited to, excipients that are found in the Cosmetics, Toiletry, Fragrance Association (CTFA) ingredient Database and the handbook of pharmaceutical excipients such as absorbents, anticaking agents, antioxidants (such as, ascorbic acid, ascorbic acid polypeptide, ascorbyl dipalmitate, BHA, BHT, magnesium ascorbate, magnesium ascorbyl phosphate, propyl gallate sodium ascorbate, sodium ascorbyl/cholesteryl phosphate, sodium bisulfite, sodium erythorbate, sodium metabisulfide, tocopheryl acetate, tocopheryl nicotinate), antistatic agents, astringents, binders, buffering agents, bulking agents, chelating agents, colorants, cosmetic astringents, biocides (such as parabens, organic acids, organic bases, alcohols. isothiazolinones and others), deodorant agents, emollients, external analgesics (such as Benzyl Alcohol, Methyl Salicylate, Camphor, Phenol, Capsaicin, Juniper Tar (Menthol, Resorcinol, Methyl Nicotinate, and Turpentine Oil), film formers, flavoring agents, fragrance ingredients, humectants, lytic agents, moisturizing agents, occlusivity enhancers, opacifying agents, oxidizing agents (such as Peroxides, Bromates, Chlorates, Potassium Iodates, and Persulfates,), reducing agents (such as Sulfites, Thioglycolates, Cystein, Cysteine HCl, Glutathione, Hydroquinone, Mercaptopropionic Acid, Sulfonates, Thioglycolic Acid), penetration enhancers, pesticides, plasticizers, preservatives, skin bleaching agents such as hydroquinone, skin conditioning agents, skin protectants (such as Allantoin, Aluminum Acetate, Dimethicone, Glycerin, Kaolin, Lanolin, Mineral Oil, Petrolatum, Talc, and Zinc Oxide), slip modifiers, solubilizing agents, solvents, sunscreen agents (such as Aminobenzoic Acid, Cinoxate, cinnamates, Aminobenzoates, Oxybenzone, Red Petrolatum, Titanium Dioxide, and Trolamine Salicylate), surface modifiers, surfactants and emulsifying agents, suspending agents, thickening agents, viscosity controlling agents including increasing or decreasing agents, UV light absorbing agents (such as Acetaminosalol, Allatoin PABA, Benzalphthalide, and Benzophenone,). Other possible excipients include, but are not limited to, sugars and derivatives (such as acacia, dextrin, dextrose, maltodextrin, and sorbitol), starch derivatives, cellulosic materials (such as methyl cellulose, Ethylcellulose, Hydroxyethylcellulose, Hydroxypropylcellulose, and Hydroxypropylmethylcellulose), polysaccharides (such as dextrates, guar gum, and xanthan gum), polyethers, suspending agents cyclodextrins, and others.

### Volatile Solvent

The formulation according to the present disclosure includes a volatile solvent. The silicone-based excipient may be contained in volatile solvent (or carrier fluid) to provide the present topical formulations. Typically, the volatile solvent is the solvent used for conducting the hydrosilylation reaction to form the silicone-based excipient. Suitable volatile solvents include volatile solvents, organic liquids (oils and solvents), silicones and mixtures thereof.

Solvents may include volatile liquids such as alcohols (e.g., methyl, ethyl, isopropyl alcohols and methylene chloride); ketones (e.g., acetone); aromatic hydrocarbons such as benzene derivatives (e.g., xylenes and toluenes); lower molecular weight alkanes and cycloalkanes (e.g., hexanes, heptanes and cyclohexanes); and alkanoic acid esters (e.g., ethyl acetate, n-propyl acetate, isobutyl acetate, n-butyl acetate, isobutyl isobutyrate, hexyl acetate, 2-ethylhexyl acetate or butyl acetate); and combinations and mixtures thereof.

Typically, the volatile solvent is an organic liquid. Organic liquids include oils and solvents. The organic liquids are exemplified by, but not limited to, aromatic hydrocarbons, aliphatic hydrocarbons, alcohols, aldehydes, ketones, amines, esters, ethers, glycols, glycol ethers, alkyl halides and aromatic halides. Hydrocarbons include, isododecane, isohexadecane, Isopar L (C11-C13), Isopar H (C11-C12), hydrogentated polydecene. Ethers and esters include, isodecyl neopentanoate, neopentylglycol heptanoate, glycol distearate, dicaprylyl carbonate, diethylhexyl carbonate, propylene glycol n-butyl ether, ethyl-3 ethoxypropionate, propylene glycol methyl ether acetate, tridecyl neopentanoate, propylene glycol methylether acetate (PGMEA), propylene glycol methylether (PGME). octyldodecyl neopentanoate, diisobutyl adipate, diisopropyl adipate, propylene glycol dicaprylate/dicaprate, and octyl palmitate. Additional volatile solvents suitable as a standalone compound or as an ingredient to the carrier fluid include fats, oils, fatty acids, and fatty alcohols.

The volatile solvent may also be a low viscosity organopolysiloxane or a volatile methyl siloxane or a volatile ethyl siloxane or a volatile methyl ethyl siloxane having a viscosity at 25° C. in the range of about 1 to about 1,000 mm²/sec, exemplified by hexamethylcyclotrisiloxane, octamethyleyelotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, hexamethyl-3,3,bis{(trimethlylsilyl)oxy}trisiloxane pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes.

### Emollients

An emollient is a hydrophobic material that provides softness, lubricity and smoothness to the skin and often forms a thin occlusive film that increases hydration by reducing transepidermal water loss (TEWL). Exemplary hydrophobic emollients include, but are not limited to, short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH; (C2-C18) alkyl or (C6-C12) aryl esters of glycerol, pentaerythritol, ethylene glycol, propylene glycol, as well as polyethoxylated derivatives of these; (C12-C22) alkyl esters or (C12-C22) ethers of polypropylene glycol; (C12-C22) alkyl esters or (C12-C22) ethers of polypropylene glycol/polyethylene glycol copolymer; and polyether polysiloxane copolymers. Additional examples of hydrophobic components include cyclic dimethicones, including volatile cyclic silicones such as D4 and D5, polydialkylsiloxanes, polyaryl/alkylsiloxanes, silicone copolyols, cocoa butter, beeswax, jojoba oil, lanolin and derivatives, long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids; hydrocarbons including straight and branched chain alkanes and alkenes such as isoparafins (e.g., isooctane, isododecane, isooctadecane, etc.), squalene, and mineral oil, polysiloxane polyalkylene copolymers, dialkoxy dimethyl polysiloxanes; (C12-C22) alkyl and (C12-C22) alkenyl alcohols, and petroleum derived alkanes such as isoparafins, petrolatum, petrolatum USP, as well as refined natural oils (especially NF or USP grades) such as olive oil NF, cotton seed oil, castor oil, peanut oil, corn oil, seasame oil, safflower oil, soybean oil, sunflower oil and the like, and blends thereof. In certain preferred embodiments, the hydrophobic components useful in the compositions of the present invention include those selected from the group consisting of petrolatum USP and short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH (such as diisopropyladipate, diisopropylsebacate); (C1-C9) alkyl or (C6-C12) aryl esters of glycerol, pentaerythritol, ethylene glycol, propylene glycol (such as glyceryl tricaprylate/caprate); and mixtures thereof. In some embodiments, the emollient is the ester of a fatty acid, e.g., a triglyceride. In some embodiments, the triglyceride is capric triglyceride or caprylic triglyceride. The esters described herein that function as permeation enhancers can also be emollients.

In some embodiments, the emollient is present in the composition from about 0% by weight to about 25% by weight. In some embodiments, the emollient is present in the composition from about 1% by weight to about 10% by weight. In some embodiments, the emollient is present in the composition from about 1% by weight to about 5% by weight. In some embodiments, the emollient is present in the composition in about 10% by weight, e.g., 10% by weight. In some embodiments, the emollient is present in the composition in about 7.5% by weight, e.g., 7.5% by weight or 7.6% by weight. In some embodiments, the emollient is present in the composition in about 5% by weight, e.g., 5% by weight. In some embodiments, the emollient is present in the composition in about 2.5% by weight, e.g., 2.5% by weight. In some embodiments, the emollient is present in the composition in about 1% by weight, e.g., 1% by weight.

### Permeation Enhancers

Compositions of the present invention can comprise one or more permeation enhancers. The permeation enhancer can be an ester or a mixture of esters. The ester can be a lactate ester, for example lauryl lactate, myristyl lactate, or cetyl lactate.

The compositions described herein can comprise a permeation enhancer, wherein the permeation enhancer is a composition comprising an α-hydroxy carboxylic acid ester and a second ester, wherein the α-hydroxy carboxylic acid ester and the second ester are not the same; and wherein less than 10% (e.g., by weight or volume) of the composition of esters comprises fatty alcohols and/or an alkyl lactyllactate and/or other related substances. In some embodiments, at least 90% (e.g., by weight or volume) of the composition is comprised of esters.

In certain embodiments, the second ester is a compound of formula (I):

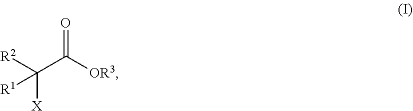

wherein each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl; each R² is independently selected from the group consisting of: H and C₁-C₂₀ alkyl; R³ is a C₁₂-C₄₀ alkyl; and X is NR¹R¹ or OR¹;

In some embodiments, the α-hydroxy carboxylic acid ester is a compound of formula (II):

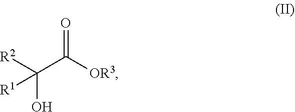

wherein R¹, R² and R³ are as defined in formula (I).

In some embodiments, the second ester is a compound of formula (II):

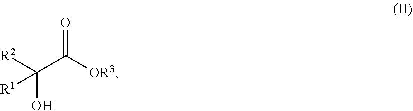

wherein R¹, R² and R³ are as defined in formula (I).

In some embodiments, the α-hydroxy carboxylic acid ester and the second esters are compounds of formula (II):

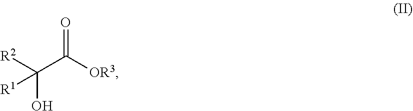

wherein R¹, R² and R³ are as described in formula (I).

In certain embodiments, the composition further comprises a third ester. In some embodiments, the composition comprises lauryl lactate, myristyl lactate, and cetyl lactate. In some embodiments, the composition further comprises a fourth ester. In some embodiments, the composition further comprises a fifth ester. In some embodiments, the composition comprises more than five or more esters.

In certain embodiments, the α-hydroxy carboxylic acid ester, the second ester and the third ester are not the same. In some embodiments, the α-hydroxy carboxylic acid ester, the second ester, the third ester and the fourth ester are not the same. In some embodiments, the second ester, the third ester, the fourth ester and the fifth are not the same.

In certain embodiments, the α-hydroxy carboxylic acid ester and the second ester used in the composition are selected from the group consisting of lauryl lactate, lauryl mandelate, myristyl lactate, cetyl lactate, stearyl lactate, ethyl hexyl lactate and dimethyl octyl lactate. In some embodiments, the α-hydroxy carboxylic acid ester, the second ester and the third ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester and the fourth ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester, the fourth ester and the fifth ester used in the composition are selected from the group consisting of lauryl lactate, myristyl lactate, cetyl lactate, stearyl lactate, ethyl hexyl lactate and dimethyl octyl lactate. In some embodiments, the esters are chosen in ratios so as to provide a preselected delivery of a drug through the skin or mucousal membrane.

In certain embodiments, the composition comprises less than 5% (e.g., by weight or volume) fatty alcohols and/or alkyl lactyllactate and/or other related substances. In certain embodiments, the composition comprises less than 3% (e.g., by weight or volume) fatty alcohols and/or alkyl lactyllactate. In certain embodiments, the composition comprises less than 1% (e.g., by weight or volume) fatty alcohols and/or alkyl lactyllactate. In some embodiments, the composition is substantially free of fatty alcohols and/or alkyl lactyllactate.

In certain embodiments, the α-hydroxy carboxylic acid ester is present in a racemic mixture (e.g., less than 10% enantiomeric excess of either the R or S stereoisomer). In some embodiments, the α-hydroxy carboxylic acid ester is present in an enantiomeric excess of the R stereoisomer (e.g., 10%, 50%, 75%, 85%, 90%, 95%, 97% or greater). In some embodiments, the α-hydroxy carboxylic acid ester is present in an enantiomeric excess of the S stereoisomer (e.g., 10%, 50%, 75%, 85%, 90%, 95%, 97% or greater).

In certain embodiments, the second ester is present in a racemic mixture (e.g., less than 10% enantiomeric excess of either the R or S stereoisomer). In some embodiments, the second ester is present in an enantiomeric excess of the R stereoisomer (e.g., 10%, 50%, 75%, 85%, 90%, 95%, 97% or greater). In some embodiments, the second ester is present in an enantiomeric excess of the S stereoisomer (e.g., 10%, 50%, 75%, 85%, 90%, 95%, 97% or greater).

In certain embodiments, the third ester is present in a racemic mixture (e.g., less than 10% enantiomeric excess of either the R or S stereoisomer). In some embodiments, the third ester is present in an enantiomeric excess of the R stereoisomer (e.g., 10%, 50%, 75%, 85%, 90%, 95%, 97% or greater). In some embodiments, the third ester is present in an enantiomeric excess of the S stereoisomer (e.g., 10%, 50%, 75%, 85%, 90%, 95%, 97% or greater). In certain embodiments, the fourth ester is present in a racemic mixture (e.g., less than 10% enantiomeric excess of either the R or S stereoisomer). In some embodiments, the fourth ester is present in an enantiomeric excess of the R stereoisomer (e.g., 10%, 50%, 75%, 85%, 90%, 95%, 97% or greater). In some embodiments, the fourth ester is present in an enantiomeric excess of the S stereoisomer (e.g., 10%, 50%, 75%, 85%, 90%, 95%, 97% or greater).

In certain embodiments, the fifth ester is present in a racemic mixture (e.g., less than 10% enantiomeric excess of either the R or S stereoisomer). In some embodiments, the fifth ester is present in an enantiomeric excess of the R stereoisomer (e.g., 10%, 50%, 75%, 85%, 90%, 95%, 97% or greater). In some embodiments, the fifth ester is present in an enantiomeric excess of the S stereoisomer (e.g., 10%, 50%, 75%, 85%, 90%, 95%, 97% or greater).

In certain embodiments, the ester is a compound of formula (I-A):

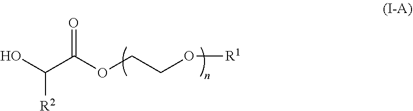

wherein
R¹ is a C₈₋₃₅ alkyl;
n is an integer from 2 to 35; and
R² is hydrogen, alkyl, aryl, heteroaryl, heterocyclyl or aralkyl;
or a pharmaceutically acceptable salt thereof.

In certain embodiments, n is an integer from 2 to 25. In some embodiments, n is an integer from 2 to 15. In some embodiments, n is an integer from 2 to 10. In some embodiments, n is an integer from 4 to 10. In some embodiments, n is 2. In some embodiments, n is 6. In some embodiments, n is 7. In some embodiments, n is 8. In some embodiments, n is 9. In some embodiments, n is 10. In some embodiments, n is 11. In some embodiments, n is 12. In certain embodiments, R¹ is a C₈₋₂₅ alkyl group. In some embodiments, R¹ is a C₈₋₂₀ alkyl group. In some embodiments, R¹ is a C₁₀₋₁₅ alkyl group. In some embodiments, R¹ is a C₁₂ alkyl group.

In certain embodiments, R² is alkyl (e.g., C₁₋₄ alkyl). In certain embodiments, R² is C₁₋₄ alkyl (e.g., methyl). In certain embodiments, R² is aryl (e.g., phenyl). In some embodiments, R² is aralkyl (e.g., benzyl).

In certain embodiments, the compound of formula (I-A) is represented by the following formula:

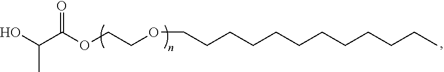

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some embodiments, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In some embodiments, the compound of formula (I-A) is represented by the following formula:

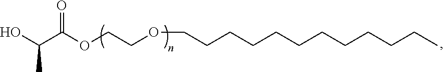

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some aspects of this embodiment, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In some embodiments, the compound of formula (I-A) is represented by the following formula:

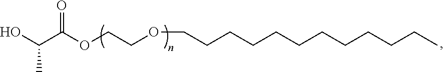

wherein n is an integer from 2 to 35. In some aspects of this embodiment, n is an integer from 2 to 25. In some aspects of this embodiment, n is an integer from 2 to 15. In some aspects of this embodiment, n is an integer from 2 to 10. In some embodiments, n is an integer from 4 to 10. In some embodiments, n is 6. In some embodiments, n is 7. In some embodiments, n is 8. In some embodiments, n is 9. In some embodiments, n is 10. In some embodiments, n is 11. In some embodiments, n is 12.

In certain embodiments, the compound of formula (I-A) may have an average molecular weight ranging from about 300 daltons to about 2000 daltons (e.g., from about 300 daltons to about 1500 daltons, from about 400 daltons to about 1200 daltons, from about 500 daltons to about 1000 daltons, from about 600 daltons to about 850 daltons).

The polymeric portion of a compound of formula (I-A) described herein may have a polymer polydispersity index (PDI) of less than or equal to about 2.5 (e.g., less than or equal to about 2.2, less than or equal to about 2.0, or less than or equal to about 1.5). In some embodiments, a hydrophobic polymer described herein may have a polymer PDI of about 1.0 to about 2.5, about 1.0 to about 2.0, about 1.0 to about 1.7, or from about 1.0 to about 1.6.

In certain embodiments, the compound of formula (I-A) is a racemic mixture (e.g., less than 10% enantiomeric excess of either the R or S stereoisomer). In certain embodiments, the compound of formula (I-A) is at least 10% enantiomeric excess of the R stereoisomer. In certain embodiments, the compound of formula (I-A) is at least 50% enantiomeric excess of the R stereoisomer. In some embodiments, the compound of formula (I-A) is at least 75% enantiomeric excess of the R stereoisomer. In some embodiments, the compound of formula (I-A) is at least 85% enantiomeric excess of the R stereoisomer. In some embodiments, the compound of formula (I-A) is at least 90% enantiomeric excess of the R stereoisomer. In some embodiments, the compound compound of formula (I-A) is at least 95% enantiomeric excess of the R stereoisomer. In some embodiments, the compound of formula (I-A) is least 97% enantiomeric excess of the R stereoisomer. In some embodiments, the compound of formula (I-A) is at least 99% enantiomeric excess of the R stereoisomer.

In certain embodiments, the formula (I-A) is at least 10% enantiomeric excess of the S stereoisomer. In certain embodiments, the compound of formula (I-A) is at least 50% enantiomeric excess of the S stereoisomer. In some embodiments, the compound of formula (I-A) is at least 75% enantiomeric excess of the S stereoisomer. In some embodiments, the compound of formula (I-A) is at least 85% enantiomeric excess of the S stereoisomer. In some embodiments, the compound of formula (I-A) is at least 90% enantiomeric excess of the S stereoisomer. In some embodiments, the compound of formula (I-A) is at least 95% enantiomeric excess of the S stereoisomer. In some embodiments, the compound of formula (I-A) is at least 97% enantiomeric excess of the S stereoisomer. In some embodiments, the compound of formula (I-A) is at least 99% enantiomeric excess of the S stereoisomer.

Other permeation enhancers may also be exemplified by monohydric alcohols such as ethanol and isopropyl, butyl and benzyl alcohols, or dihydric alcohols such as ethylene glycol, diethylene glycol, or propylene glycol, dipropylene glycol and trimethylene glycol, or polyhydric alcohols such as butylene glycol, hexylene glycol, polypropylene glycol, ethylene glycol, and polyethylene glycol, which enhance drug solubility; polyethylene glycol ethers of aliphatic alcohols (such as cetyl, lauryl, oleyl and stearyl) including polyoxyethylene (4) lauryl ether, polyoxyethylene (2) oleyl ether and polyoxyethylene (10) oleyl ether commercially available under the trademark BRIJ® 30, 93 and 97, respectively, from Uniqema Americas LLC (Wilmington, Del.), and others such as BRIJ® 35, 52, 56, 58, 72, 76, 78, 92, 96, 700 and 721; vegetable, animal and fish fats and oils such as olive, and castor oils, squalene, lanolin; fatty acids such as oleic, linoleic, and capric acid, and the like; fatty acid esters such as propyl oleate, decyl oleate, isopropyl palmitate, glycol palmitate, glycol laurate, dodecyl myristate, isopropyl myristate and glycol stearate which enhance drug diffusibility; fatty acid alcohols such as oleyl alcohol and its derivatives; fatty acid amides such as oleamide and its derivatives; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide and dimethylformamide which affect keratin permeability; salicylic acid; amino acids; benzyl nicotinate; and higher molecular weight aliphatic surfactants such as lauryl sulfate salts; and esters of sorbitol and sorbitol anhydride such as polysorbate 20 commercially available under the trademark Tween® 20 from Uniqema Americas LLC (Wilmington, Del.), as well as other polysorbates such as 21, 40, 60, 61, 65, 80, 81, and 85. Other enhancers include enzymes, panthenol, and other non-toxic enhancers commonly used in transdermal or transmucosal compositions.

Polyhydric alcohols also include glycols, triols and polyols having 4 to 6 alcoholic hydroxyl groups. Typical of said glycols are glycols containing 2 to 6 carbon atoms, e.g. ethylene glycol, propylene glycol, butylene glycol, polyethylene glycol (average molecular weight about 200-8,000, preferably about 200 to 6,000), etc. Examples of said triols include glycerin, trimethylolpropane, etc. Said polyols are exemplified by sorbitol, polyvinylpyrrolidone, etc. These polyhydric alcohols may be used either singularly or in combination (preferably, of two or three). Thus, for example, glycerin or dipropylene glycol alone, or a mixture of either glycerin or dipropylene glycol with butylene glycol can be employed.

In some embodiments, the permeation enhancer is present in the composition from about 0% by weight to about 25% by weight. In some embodiments, the permeation enhancer is present in the composition from about 1% by weight to about 10% by weight. In some embodiments, the permeation enhancer is present in the composition from about 1% by weight to about 5% by weight. In some embodiments, the permeation enhancer is present in the composition in about 10% by weight, e.g., 10% by weight. In some embodiments, the permeation enhancer is present in the composition in about 7.5% by weight, e.g., 7.5% by weight or 7.6% by weight. In some embodiments, the permeation enhancer is present in the composition in about 5% by weight, e.g., 5% by weight. In some embodiments, the permeation enhancer is present in the composition in about 2.5% by weight, e.g., 2.5% by weight. In some embodiments, the permeation enhancer is present in the composition in about 1% by weight, e.g., 1% by weight.

### Essential Oils

The compositions of the present invention can comprise one or more essential oils. Non-limiting examples of essential oils include birch oil, lavender oil, eucalyptus oil, peppermint oil, and a high terpene oil. In a preferred embodiment, a composition described herein comprises birch oil, e.g., birch oil derived from the bark of Betula lenta.

In some embodiments, the essential oil is present in the composition from about 0% by weight to about 25% by weight. In some embodiments, the essential oil is present in the composition from about 1% by weight to about 10% by weight. In some embodiments, the carrier oil is present in the composition from about 1% by weight to about 5% by weight. In some embodiments, the carrier oil is present in the composition in about 10% by weight, e.g., 10% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Viscosity Enhancement Agent

The compositions of the present invention can comprise one or more viscosity enhancement agents. A non-limiting example of a viscosity enhancement agent includes silica silylate. The viscosity enhancement agent can also be used to retain the fragrance of a cosmetic composition after application onto a keratinous surface.

In some embodiments, the viscosity enhancement agent is present in the composition from about 0.1% by weight to about 3% by weight. In some embodiments, the viscosity enhancement agent is present in the composition from about 0.1% by weight to about 1% by weight. In some embodiments, the viscosity enhancement agent is present in the composition in about 0.2% by weight, e.g., 0.2% by weight. In some embodiments, the carrier oil is present in the composition in about 7.5% by weight, e.g., 7.5% by weight or 7.6% by weight. In some embodiments, the carrier oil is present in the composition in about 5% by weight, e.g., 5% by weight. In some embodiments, the carrier oil is present in the composition in about 2.5% by weight, e.g., 2.5% by weight. In some embodiments, the carrier oil is present in the composition in about 1% by weight, e.g., 1% by weight.

### Active Ingredients

The formulation may include an active ingredient (or “active”) selected from any personal, healthcare, and pharmaceutical active. As used herein, a “personal care active” means any compound or mixtures of compounds that are known in the art as additives in the personal care formulations that are typically added for treating hair or skin to provide a cosmetic and/or aesthetic benefit. A “healthcare active” means any compound or mixtures of compounds that are known in the art to provide a pharmaceutical or medical benefit. Thus, “healthcare active” includes materials considered as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, mitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

In some embodiments, the active is present in the composition from about 0% by weight to about 25% by weight. In some embodiments, the active is present in the composition from about 1% by weight to about 10% by weight. In some embodiments, the active is present in the composition from about 1% by weight to about 5% by weight. In some embodiments, the active is present in the composition in about 10% by weight, e.g., 10% by weight. In some embodiments, the active is present in the composition in about 7.5% by weight, e.g., 7.5% by weight or 7.6% by weight. In some embodiments, the active is present in the composition in about 5% by weight, e.g., 5% by weight. In some embodiments, the active is present in the composition in about 2.5% by weight, e.g., 2.5% by weight. In some embodiments, the active is present in the composition in about 1% by weight, e.g., 1% by weight.

### Cannabinoids

Cannabinoids are a group of chemicals known to activate cannabinoid receptors in cells. The compositions of the present invention can comprise one or more cannabinoids. In some embodiments, the cannabinoid is derived from a naturally occurring source, e.g., a natural oil from a plant. In some embodiments, the cannabinoid is a synthetic cannabinoid, e.g., synthetic cannabidiol. As used herein, a “synthetic cannabinoid” refers to a naturally occurring cannabinoid or a cannabinoid that is not naturally occurring that has been chemically synthesized using methods of synthetic organic chemistry. As used herein, “synthetic cannabidiol” refers to cannabidiol that has been chemically synthesized using methods of synthetic organic chemistry. Synthetic cannabinoids may be synthesized using conventional methods known to one skilled in the art.

In some embodiments, the composition, e.g., an anhydrous gel, comprises a naturally occurring oil comprising a cannabinoid, e.g., an oil derived from a cannabis plant. In some embodiments, the naturally occurring oil is present in the composition from about 1% by weight to about 50% by weight. In some embodiments, the naturally occurring oil is present in the composition from about 1% by weight to about 40% by weight. In some embodiments, the naturally occurring oil is present in the composition from about 1% by weight to about 30% by weight. In some embodiments, the naturally occurring oil is present in the composition from about 1% by weight to about 20% by weight. In some embodiments, the naturally occurring oil is present in the composition from about 1% by weight to about 10% by weight. In some embodiments, the naturally occurring oil is present in the composition from about 1% by weight to about 5% by weight. In some embodiments, the naturally occurring oil is present in the composition in about 10% by weight, e.g., 10% by weight. In some embodiments, the naturally occurring oil is present in the composition in about 5% by weight, e.g., 5% by weight. In some embodiments, the naturally occurring oil is present in the composition in about 2.5% by weight, e.g., 2.5% by weight. In some embodiments, the naturally occurring oil is present in the composition in about 1% by weight, e.g., 1% by weight.

In some embodiments, the composition, e.g., an anhydrous gel, comprises a cannabinoid that is not in a naturally occurring oil, e.g., a synthetic cannabinoid or cannabinoid isolated from a naturally occurring oil. In some embodiments, the cannabinoid is present in the composition from about 1% by weight to about 50% by weight. In some embodiments, the cannabinoid is present in the composition from about 1% by weight to about 40% by weight. In some embodiments, the cannabinoid is present in the composition from about 1% by weight to about 30% by weight. In some embodiments, the cannabinoid is present in the composition from about 1% by weight to about 20% by weight. In some embodiments, the cannabinoid is present in the composition from about 1% by weight to about 10% by weight. In some embodiments, the cannabinoid is present in the composition from about 1% by weight to about 5% by weight. In some embodiments, the cannabinoid is present in the composition in about 10% by weight, e.g., 10% by weight. In some embodiments, the cannabinoid is present in the composition in about 5% by weight, e.g., 5% by weight. In some embodiments, the cannabinoid is present in the composition in about 2.5% by weight, e.g., 2.5% by weight. In some embodiments, the cannabinoid is present in the composition in about 1% by weight, e.g., 1% by weight. In some embodiments, the cannabinoid is present in the composition in about 0.5% by weight, e.g., 0.5% by weight.

The cannabinoids described herein may also be derived from other sources not described above, such as cannabinoids produced by a genetically-modified bacterium. In some embodiments, the cannabinoid is present in the composition from about 1% by weight to about 50% by weight. In some embodiments, the cannabinoid is present in the composition from about 1% by weight to about 40% by weight. In some embodiments, the cannabinoid is present in the composition from about 1% by weight to about 30% by weight. In some embodiments, the cannabinoid is present in the composition from about 1% by weight to about 20% by weight. In some embodiments, the cannabinoid is present in the composition from about 1% by weight to about 10% by weight. In some embodiments, the cannabinoid is present in the composition from about 1% by weight to about 5% by weight. In some embodiments, the cannabinoid is present in the composition in about 10% by weight, e.g., 10% by weight. In some embodiments, the cannabinoid is present in the composition in about 5% by weight, e.g., 5% by weight. In some embodiments, the cannabinoid is present in the composition in about 2.5% by weight, e.g., 2.5% by weight. In some embodiments, the cannabinoid is present in the composition in about 1% by weight, e.g., 1% by weight. In some embodiments, the cannabinoid is present in the composition in about 0.5% by weight, e.g., 0.5% by weight.

“Cannabinoid,” as used herein, is meant to include compounds which interact with the cannabinoid receptor and various cannabinoid mimetics, such as certain tetrahydropyran analogs (e.g., Δ⁹-tetrahydrocannabinol, Δ⁸-tetrahydrocannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylhept-yl, (+)-(3S,4S)-7-hydroxy-Δ⁶-tetrahydrocannabinol-1,1-dimethylh-eptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1-,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenedi-of and 4-(1,1-dimethylheptyl)-2,3′-dihydroxy-6′alpha-(3-hydroxypropyl)-1′,-2′,3′,4′,5′,6′-hexahydrobiphenyl), as well as their pharmaceutically acceptable salts, solvates, metabolites (e.g., cutaneous metabolites), and metabolic precursors. Further examples of “cannabinoids” include those compounds described in the references cited below.

“Δ⁹-THC,” as used herein, is meant to refer to Δ⁹-tetrahydrocannabinol as well as to its pharmaceutically acceptable salts, solvates, metabolites (e.g., cutaneous metabolites), and metabolic precursors. Δ⁹-tetrahydrocannabinol is marketed under the generic name “dronabinol.”

“Cannabinol,” as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6H-dibenzo[b,d]pyran-1-ol. The synthesis of 6,6,9-trimethyl-3-pentyl-6H-dibenzo[b,d]pyran-1-ol is described in, for example, Novak et al., Tetrahedron Letters, 23:253 (1982), which is hereby incorporated by reference.

“Cannabidiol,” as used herein, is meant to refer to 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenediol. The synthesis of 2-[3-methyl-6-(1-methylethenyl)-2-cyclohexen-1-yl]-5-pentyl-1,3-benzenediol is described, for example, in Petilka et al., Helv. Chim. Acta, 52:1102 (1969) and in Mechoulam et al., J. Am. Chem. Soc., 87:3273 (1965), which are hereby incorporated by reference.

“Nabilone,” as used herein, is meant to refer to 3-(1,1-dimethylheptyl)-6,6a,7,8,10,10a-hexahydro-1-hydroxy-6,6-dimethyl-9-H-dibenzo[b,d]pyran-9-one as well as to pharmaceutically acceptable salts, solvates, metabolites (e.g., cutaneous metabolites), and metabolic precursors of 3-(1,1-dimethylheptyl)-6,6a,7,8,10,10a-hexahydro-1-hydroxy-6,6-dimethyl-9H-dibenzo[b,d]pyran-9-one. 3-(1,1-dimethylheptyl)-6,6a,7,8,10,10a-hexahydro-1-hydroxy-6,6-dimethyl-9-H-dibenzo[b,d]pyran-9-one is approved for use in the United Kingdom for treating nausea and vomiting associated with chemotherapy, and its preparation is described, for example, in U.S. Pat. No. 3,968,125 to Archer, which is hereby incorporated by reference.

“Levonantradol,” as used herein, is meant to refer to (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate, as well as to pharmaceutically acceptable salts, solvates, metabolites (e.g., cutaneous metabolites), and metabolic precursors of (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate. (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate is particularly useful in pain control, and its synthesis is described in Belgian Pat. No. 854,655, which is hereby incorporated by reference; in U.S. Pat. Nos. 4,206,225, 4,232,018, and 4,260,764, each to Johnson, which are hereby incorporated by reference; in U.S. Pat. No. 4,235,913 to Johnson et al., which is hereby incorporated by reference; in U.S. Pat. No. 4,243,674 to Bindra, which is hereby incorporated by reference; and in U.S. Pat. Nos. 4,263,438, 4,270,005, and 4,283,569, each to Althuis et al., which are hereby incorporated by reference.

“(−)-HU-210,” as used herein, is meant to refer to (−)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (−)-(3S,4S)-7-hydroxy-Δ⁶-tetrahydrocannabinol-1,1-dimethylheptyl. (−)-(3S,4S)-7-hydroxy-Δ⁶-tetrahydrocannabinol-1,1-dimethylheptyl is particularly useful in pain control, and its preparation is described in U.S. Pat. Nos. 4,876,276 and 5,521,215, each to Mechoulam et al., which are hereby incorporated by reference.

“(+)-HU-210,” as used herein, is meant to refer to (+)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (+)-(3S,4S)-7-hydroxy-Δ⁶-tetrahydrocannabinol-1,1-dimethylheptyl. (+)-(3S,4S)-7-hydroxy-Δ⁹-tetrahydrocannabinol-1,1-dimethylheptyl is sometimes referred to as HU-211 and/or dexanabinol; it is an antagonist of the N-methyl-D-aspartate receptor; and its preparation is described in U.S. Pat. Nos. 4,876,276 and 5,521,215, each to Mechoulam et al., which are hereby incorporated by reference.

“11-hydroxy-Δ⁹-THC,” as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahydrocannabinol is a more hydrophilic, psychoactive metabolite of Δ⁹-tetrahydrocannabinol, and its laboratory synthesis has been described in Siegel et al., J. Org. Chem., 54:5428 (1989), which is hereby incorporated by reference.

“Δ⁸-THC-11-oic acid,” as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabinol-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa) and is produced from 6a,7,10,10a-tetrahydro-6,6,9-trimethyl-3-pentyl-6H-dibenzo[b,d]pyran-l-o-1 via a series of biotransformations mediated primarily by mammalian liver enzymes. Δ⁸-tetrahydrocannabinol-11-oic acid can also be produced synthetically by reference to the synthetic schemes set forth in U.S. Pat. No. 6,162,829 to Burstein, which is hereby incorporated by reference. Δ⁸-tetrahydrocannabin-ol-11-oic acid is more hydrophilic than 6a,7,10,10a-tetrahydro-6,6,9-trimethyl-3-pentyl-6H-dibenzo[b,d]pyran-1-ol-, and it has analgesic activity.

“CP 55,940,” as used herein, refers to 4-(1,1-dimethylheptyl)-2,3′-dihydroxy-6′alpha-(3-hydroxypropyl)-1′,2′,3′,-4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′alpha-(3-hydroxypropyl)-1′,2′,3′,-4′,5′,6′-hexahydrobiphenyl is sometimes referred to as (−)-cis-3-[2-Hydroxy-4-(1,1-dimethylheptyl)phenyl]-trans-4-(3-hydroxypropyl)cyclohexanol, and it is commercially available from Tocris Cookson, Inc., Ellisville, Mo. Its preparation has been described in U.S. Pat. No. 4,371,720 to Johnson et al. and U.S. Pat. No. 4,663,474 to Urban, which are hereby incorporated by reference.

“R(+)-WIN 55,212-2,” as used herein, refers to (R)-(+)-[2,3-dihydro-5-methyl-3-(4-morpholinylmethyl)pyrrolo[1,2,3-de]-1,4-benzoxazin-6-yl]-1-naphthalenylmethanone, as well as to its pharmaceutically acceptable salts, solvates, metabolites (e.g., cutaneous metabolites), and metabolic precursors. (R)-(+)-[2,3-dihydro-5-methyl-3-(4-morpholinylmethyl)-pyrrolo[1,2,3-de]-1-,4-benzoxazin-6-yl]-1-naphthalenyl-methanone (in its mesylate form) is commercially available, for example, from Tocris Cookson, Inc., Ellisville, Mo., and from Research Biochemicals International, Natick, Mass.

The compositions described herein can further include one or more additional cannabinoids. The one or more additional cannabinoids can be selected from the aforementioned list of cannabinoids or it (they) can be selected from cannabinoids which are not contained in the aforementioned list, such as Δ⁸-THC, high affinity cannabinoid receptor agonists (other than R(+)-WIN 55,212-2 and CP 55,940), and the like. Illustratively, the cannabinoid composition can include two or more cannabinoids, each being selected from the group consisting of Δ⁹-THC, cannabinol, cannabidiol, nabilone, levonantradol, (−)-HU-210, (+)-HU-210, 11-hydroxyΔ⁹-THC, Δ⁸-THC-11-oic acid, CP 55,940, and R(+)-WIN 55,212-2.

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 to Martin et al., which is hereby incorporated by reference.

“Metabolites” of cannabinoids, as used herein, are meant to include compounds which are produced by the metabolic processes (e.g., cutaneous metabolic processes and/or systemic metabolic processes) of the subject's body. Suitable metabolites can be identified, for example, by studying the kinetics of drug enzymatic metabolism in skin homogenates. Illustratively, skin homogenates can be prepared from 250-μm dermatomed fresh healthy abdominal plastic surgery samples. The skin is homogenized (e.g., using a Polytron tissue homogenizer and ground glass homogenizer fitted with a glass pestle) in 4-(2-hydroxyethyl)-1-piperazineethanesulfonic acid (“HEPES”)-buffered Hanks' balanced salt solution. Whole homogenates can be used for these studies or, if significant mitochondrial or nuclear metabolism is found not to occur (e.g., by comparing the degree of metabolism in the supernatant to the degree of metabolism in the whole homogenate), the studies can be carried out on only the supernatant fraction. The drug (solubilized in, for example, buffer, ethanol, dimethylsulfoxide, or combinations thereof) is then incubated with the homogenate (or supernatant) along with NADPH (or a generating system), NADH, MgCl₂, and bovine serum albumin. The total volume of ethanol in the reaction mixture should be small (e.g., under 2%) to help minimize ethanol's detrimental effects on the enzymes. After incubating for a period of time, the reaction is terminated with 15% trichloroacetic acid, and the drug and its metabolites are obtained by solid-phase extraction. The metabolite or metabolites formed can then be identified and assayed by any suitable method (e.g., HPLC).

### Occlusivity Agent

The formulation may include an occlusivity agent configured to provide occlusivity when the formulation is applied on top of the skin. The occlusivity agent may include petrolatum, organic wax, silicone wax, polyacrylates and methacrylates (exemplified by, but not limited to Eudragit® E100, 5100, L100, and L100-55), polyvinyl pyrolidone, polyvinyl alcohol, vinylacetate-vinylpyrolidone copolymer, or any combination thereof. A majority of film-forming polymers can be considered to provide occlusive properties to the formulation and thus any suitable film-forming polymer may be used in the present formulation.

The occlusivity agent may be a wax or a wax-like material. The waxes or wax-like materials useful in the formulation according to the present disclosure generally have a melting point range of about 35 to 120° C. at atmospheric pressure. Waxes in this category include synthetic wax, ceresin, paraffin, ozokerite, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or mixtures thereof. Additionally, the occlusivity agent may include waxes capable of being used as non-silicone fatty substances, animal waxes, such as beeswax; vegetable waxes, such as carnauba, candelilla wax; mineral waxes, such as paraffin or lignite wax; microcrystalline waxes; ozokerites; synthetic waxes, including polyethylene waxes, and waxes obtained by the Fischer-Tropsch synthesis. Additionally, the occlusivity agent may include silicone waxes, polymethylsiloxane alkyls, alkoxys and/or esters.

### Additional Optional Components

The formulation may also contain a number of optional ingredients. In particular, these optional components are selected from those known in the art to be ingredients used in personal care or pharmaceutical formulations. Illustrative, non-limiting examples include surfactants, solvents, powders, coloring agents, thickeners, waxes, gelling agents or clays, stabilizing agents, pH regulators, silicones, or other suitable agents.

A thickening agent may be added to provide a desired or convenient viscosity. For example, viscosities within the range of 500 to 25,000 mm²/s at 25° C. Alternatively, thickening agents may be added to obtain viscosities within the range of about 3,000 to about 7,000 mm²/s. Suitable thickening agents are exemplified by sodium alginate, gum arable, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or mixtures of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent may be present in an amount from about 0.05 to about 10% by weight, or, alternatively about 0.05 to about 5% by weight based on the total weight of the formulation.

Also, various cosmetic, personal care, and cosmetic components may be included aside from the excipient or excipients. Examples of suitable cosmetic, and personal care components include, but are not limited to, alcohols, fatty alcohols and polyols, aldehydes, alkanolamines, alkoxylated alcohols butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, TiO₂, ZnO), ketones (e.g. camphor), isethionates, lanolin and derivatives, organic salts, phenols including salts phosphorus compounds (e.g. phosphate derivatives), polyacrylates and acrylate copolymers, synthetic polymers including salts, siloxanes and silanes, sorbitan derivatives, sterols, sulfonic acids and derivatives and waxes.

Other additives can include powders and pigments. The powder component that may be included can be generally defined as dry, particulate matter having an average particle size of about 0.02-50 microns. The particulate matter may be colored or non-colored (for example, white). Suitable powders include, but are not limited to, bismuth oxychloride, titanated mica, fumed silica, spherical silica beads, polymethylmethacrylate beads. The above mentioned powders may be surface treated to render the particles hydrophobic in nature.

The powder component also may also include various organic and inorganic pigments. The organic pigments are generally various aromatic types including azo, indigoid, triphenylmethane, anthraquinone, and xanthine dyes. Inorganic pigments generally consist of insoluble metallic salts of certified color additives, referred to as the Lakes or iron oxides. A pulverulent coloring agent, such as carbon black, and titanium dioxide, pearlescent agents, generally used as a mixture with colored pigments, or some organic dyes, generally used as a mixture with colored pigments and commonly used in the cosmetics industry, can be added to the formulation. In general, these coloring agents can be present in an amount by weight from about 0 to 20% with respect to the weight of the final formulation.

Pulverulent inorganic or organic fillers can also be added, generally in an amount by weight from about 0 to about 40% with respect to the weight of the final formulation. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, and silicone resin microbeads.

Optional components included in the present formulation may also include other silicones (including any already described above), organofunctional siloxanes, alkylmethylsiloxanes, siloxane resins and silicone gums.

The topical formulations according to the present disclosure may be in the form of a cream, a gel, a powder, a paste, or a freely pourable liquid. Generally, such formulations can generally be prepared at room temperature if no solid materials at room temperature are presents in the formulations, using simple propeller mixers, Brookfield counter-rotating mixers, or homogenizing mixers. No special equipment or processing conditions are typically required. Depending on the type of form made, the method of preparation will be different, but such methods are well known by those of ordinary skill in the art.

Anhydrous formulations may be prepared without the addition of any preservatives.

In embodiments where the substrate is skin, the formulation is applied to the skin. The skin may be healthy and intact, or it may be damaged or wounded. The formulation may be applied, i.e., rubbed or coated, directly onto the skin. Alternatively, the formulation may be deposited on a transdermal patch prior to application of the formulation to the substrate, i.e., to the skin.

The formulations according to the present disclosure can be used by standard and well-known methods, such as applying them to the human body, e.g. skin, hair, or teeth, using applicators, brushes, applying by hand, pouring them and/or possibly rubbing or massaging the formulation onto or into the body. Removal methods are also well known standard methods, including washing, wiping, peeling and the like. According to some embodiments, no removal of the formulation is required as the formulation is fully absorbed into the skin, such that no residue remains on the skin. An effective amount of the formulation for the particular purpose is applied to the skin. Such effective or therapeutic amounts generally range from about 1 mg/cm² to about 10 mg/cm². Application to the skin typically includes working the formulation into the skin. This method for applying to the skin comprises the steps of contacting the skin with the formulation in an effective amount and then rubbing the formulation onto the skin. These steps can be repeated as many times as desired to achieve the desired benefit.

### Methods of Treatment

Also described herein are methods of treating a disorder in a subject in need thereof, comprising administering to the subject a composition described herein, e.g., a composition comprising a cannabinoid and a silicone elastomer. Exemplary disorders that can be treated by the methods of the present invention include, but are not limited to, nausea, pain, epilepsy, refractory spasticity, anorexia, neuropathic pain, inflammation, Crohns disease, gout, opioid dependence, insomnia, psoriasis, shingles, rheumatoid arthritis, migraine, Dravet syndrome, glaucoma, and cancer. In some embodiments, the pain is nociceptive pain. In some embodiments, the pain is neuropathic pain. In some embodiments, the pain is psychogenic pain.

The therapeutic agent in the compositions described herein can be a cannabinoid. The clinical usefulness of cannabinoids, including cannabidiol, to provide analgesia and neuroprotection, reduce inflammation, help alleviate nausea and emesis, as well as treat epilepsy, anxiety disorders and glaucoma, has been well-recognized. In addition, it is also well-known that cannabidiol lacks the psychoactive effects seen in many of the other cannabinoids, including Δ⁹-tetrahydrocannabinol, which is currently available in an oral dosage form, sold under the trade name Marinol®.

Pain is the most frequently reported symptom and it is a common clinical problem confronting all clinicians. Millions of people in the United States suffer from severe pain that, according to numerous recent reports, is chronically under-treated or inappropriately managed. Similarly, millions of people also suffer from severe nausea and/or frequent emesis. Moreover, all too frequently, many patients suffering from chronic, under-treated or irretraceable pain also suffer from lack of appetite, nausea and/or frequent emesis. These patients present a greater clinical challenge as they are unable to receive effective doses of oral pain medications, thereby leaving their pain unalleviated. Cannabinoids, including cannabidiol, are effective in alleviating pain. Moreover, cannabinoids, including cannabidiol, can reduce a patient's nausea and vomiting, independent of any pain relief achieved. Thus, cannabinoids are particularly useful in patients experiencing nausea and vomiting secondary to un- or under-treated pain.

### Pain

Pain is one of the most common reasons for a patient to seek medical care and in consequence, pain results in a tremendous number of lost work days per year. There are three general classes of pain: nociceptive pain, neuropathic pain, and psychogenic pain.

In nociceptive pain, the stimulation of the sensory nerve endings called nociceptors cause the sensation of pain. Such pain often occurs after injury or surgery. The pain signals are transmitted by the nociceptors to the brain. Often the pain is localized, constant and has an aching or throbbing quality. Once the damage to the tissue heals, the pain usually resolves. Treatment with opioids often resolves nociceptive pain.

Psychogenic pain is a pain disorder that is associated with psychological factors. Some types of mental or emotional problems can cause pain. They can also increase or prolong pain. Headaches, muscle pains, back pain, and stomach pains are some of the most common types of psychogenic pain. People with this pain disorder actually have real pain. The diagnosis is made when all physical causes of pain are ruled out.

Neuropathic pain is the result of an injury or malfunction of the peripheral or the central nervous system. The pain may be triggered by an injury but not necessarily by an injury of the nervous system itself. Neuropathic pain is frequently chronic and is often refractory to treatment with opioids.

Neuropathic pain is caused by abnormalities in the nerves, spinal cord or brain and is a chronic type of non-malignant pain with an estimated prevalence of over 1% of the population. Optimising pain relief in these patients is crucial in helping a patient regain control of his or her life.

The most common cause of neuropathic pain is injury or dysfunction of nerves. Injury or dysfunction of peripheral nerves or nerves descending from the spinal cord results in disinhibition of nerve impulses at the spinal cord which in consequence results in pain. Neuropathic pain can also be centrally mediated, rather than peripheral, in conditions such as spinal cord injury and multiple sclerosis.

Neuropathic pain can therefore be sub-divided into two further classes; peripheral neuropathic pain and central neuropathic pain depending on whether the peripheral or central nervous system is affected.

Patients with peripheral neuropathic pain often experience pain which feels like a burning or electrical pain, whereas others describe their pain as feeling like extreme cold or pins and needles. The pain may be worsened by activity or by wearing clothes over the affected area. The pain may also follow a daily pattern, which may mean it is worse at certain times of the day.

Allodynia is a type of peripheral neuropathic pain. This is a painful response to a typically non-painful stimulus, for example brushing the affected area with a fingertip. The pain tends to increase with repeated stimulation and may spread from the affected area. Allodynic pain can be evoked in response to chemical, thermal (cold or heat) or mechanical low or high intensity stimuli applied either statically or dynamically to skin, joints, bone, muscle or viscera. It is thought that the presence of allodynic pain is a more suitable means of grouping patients suffering from peripheral neuropathic pain than by the specific disease that led to the neuropathic pain.

It is clear that patients who suffer from neuropathic pain can have their quality of life greatly affected by it. The pain can interfere with work and social activities as well as with the amount and quality of sleep that a patient experiences. A successful treatment for the relief of neuropathic pain should improve both the amount of pain that the patient is experiencing as well as improving the patient's quality of life.

The use of pharmaceutical medicaments is the most common treatment for neuropathic pain. Analgesics, antidepressants and anticonvulsants are the drug classes generally in use. The drug carbamezepine, which is an anticonvulsant, is currently the only FDA approved drug which has an indication for neuropathic pain. It has been suggested in post-marketing studies that there is a five- to eight-fold increase in the risk of blood dyscrasias in patients taking carbamezepine. In 7% of patients there has been shown to be a 25% decrease in their white blood cell count.

The use of cannabis as a medicine has long been known and during the 19^{th} Century, preparations of cannabis were recommended as a hypnotic sedative which were useful for the treatment of hysteria, delirium, epilepsy, nervous insomnia, migraine, pain and dysmenorrhoea.

### EXAMPLES

The compositions of the present invention may be prepared using methodology that is well known by an artisan of ordinary skill, e.g., by using established mixing/blending procedures, whereby the ingredient(s) to be incorporated are simply added to the composition, e.g., the anhydrous gel.

The following is a description of the manufacture of specific gels and compositions of the present invention. These examples are non-limiting and other gels and compositions of the invention can be prepared in an analogous manner by a person of ordinary skill in the art.

### Example 1: Anhydrous Cosmetic Base Gel A

TABLE 1

| Ingredient | Weight % |
|---|---|
| Isododecane (and) dimethicone/bis-isobutyl | 67.40 |
| PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Sunflower seed oil | 15.00 |
| Silica silylate | 0.55 |
| | 100.00: |

### Example 2: Anhydrous Cosmetic Base Gel B

TABLE 2

| Ingredient | Weight % |
|---|---|
| Isododecane (and) dimethicone/bis-isobutyl | 65.00 |
| PPG-20 crosspolymer (85:15) | |
| Caprylic/capric triglyceride | 8.50 |
| Phenyl trimethicone | 8.30 |
| Chrystaphyl ® (lauryl lactate) | 1.00 |
| Myristly lactate | 2.00 |
| Sunflower seed oil | 14.90 |
| Silica silylate | 0.30 |
| | 100.00 |

### Example 3: Anhydrous Cosmetic Base Gel C

TABLE 3

| Ingredient | Weight % |
|---|---|
| Isododecane (and) dimethicone/bis-isobutyl | 65.00 |
| PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Myristyl lactate | 1.75 |
| Cetyl lactate | 3.00 |
| Sunflower seed oil | 12.90 |
| Silica silylate | 0.30 |
| | 100.00 |

### Example 4: Anhydrous Cosmetic Base Gel D

TABLE 4

| Ingredient | Weight % |
|---|---|
| Isododecane (and) dimethicone/bis- | 55.00 |
| isobutyl PPG-20 crosspolymer (85:15) | |
| Dimethicone (and) dimethicone/vinyl dimethicone | 10.00 |
| crosspolymer (and) dimethicone crosspolymer (and) | |
| beeswax (and) silica (and) silica silylate | |
| Caprylic/capric triglyceride | 6.50 |
| Coco-caprylate/caprate | 2.00 |
| Phenyl trimethicone | 8.30 |
| Chrystaphyl ® (lauryl lactate) | 2.00 |
| Myristyl lactate | 3.00 |
| Sunflower seed oil | 12.90 |
| Silica silylate | 0.30 |
| | 100.00 |

### Example 5: Anhydrous Cosmetic Base Gel E

TABLE 5

| Ingredient | Weight % |
|---|---|
| Isododecane (and) dimethicone/bis- | 65.00 |
| isobutyl PPG-20 crosspolymer (85:15) | |
| Caprylic/capric triglyceride | 8.50 |
| Phenyl trimethicone | 8.30 |
| Chrystaphyl ® (lauryl lactate) | 0.25 |
| Camphor | 5.00 |
| Sunflower seed oil | 12.65 |
| Silica silylate | 0.30 |
| | 100.00 |

### Example 6: Anhydrous Cosmetic Base F with Birch Oil

TABLE 6

| Ingredient | Weight % |
|---|---|
| Isododecane (and) dimethicone/bis- | 67.00 |
| isobutyl PPG-20 crosspolymer (85:15) | |
| Caprylic/capric triglyceride | 8.50 |
| Phenyl trimethicone | 8.25 |
| Chrystaphyl ® (lauryl lactate) | 0.25 |
| Myristyl lactate | 2.75 |
| Betula lenta (bark) oil | 5.00 |
| Sunflower seed oil | 8.00 |
| Silica silylate | 0.25 |
| | 100.00 |

### Example 7: Anhydrous Mentholated Gel (Compare to Vicks VapoRub®)

TABLE 7

| Ingredient | Weight % |
|---|---|
| Anhydrous Camphorated Base E | 93.80 |
| Peppermint oil | 5.00 |
| Eucalyptus oil | 1.20 |
| | 100.00 |

### Example 8: Cannabidiol (CBD) Oil-Containing Gel

TABLE 8

| Ingredient | Weight % |
|---|---|
| Anhydrous Cosmetic Base Gel B | 94.50 |
| CBD oil | 5.00 |
| Fragrance | 0.50 |
| | 100.00 |

### Example 9: Cannabis Oil-Containing Gel

TABLE 9

| Ingredient | Weight % |
|---|---|
| Anhydrous Cosmetic Base Gel F | 95.00 |
| Cannabis oil | 5.00 |
| | 100.00 |

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Detailed Description, but rather is as set forth in the appended claims. Those of ordinary skill in the art will appreciate that various changes and modifications to this description may be made without departing from the spirit or scope of the present invention, as defined in the following claims.

1. A composition comprising a cannabinoid, a silicone elastomer, and silica silylate and is formulated for topical or transdermal administration.

2. The composition of claim 1, wherein the composition is anhydrous.

3. The composition of claim 1, wherein the silicone elastomer is present in the composition at 25% by weight or less.

4. The composition of claim 1, wherein the silicone elastomer is a dimethicone/bis-isobutyl polypropylene glycol crosspolymer.

5. The composition of claim 3, wherein the silicone elastomer is dimethicone/bis-isobutyl PPG-20 crosspolymer.

6. The composition of claim 1, further comprising isododecane.

7. The composition of claim 6, wherein the weight ratio of isododecane to bis-isobutyl PPG-20 crosspolymer is about 85:15.

8. The composition of claim 1, wherein the composition further comprises an oil.

9. The composition of claim 8, wherein the oil is sunflower seed oil, peppermint oil, eucalyptus oil, or cannabis oil.

10. The composition of claim 9, comprising a plurality of oils.

11. The composition of claim 10, wherein the composition comprises from about 10 to about 20% by weight of oil.

12. The composition of claim 1, wherein the composition comprises from about 14 to about 16% by weight of oil.

13. The composition of claim 1, wherein the composition further comprises a permeation enhancer.

14. The composition of claim 13, wherein the permeation enhancer is selected from the group consisting of lauryl lactate, myristyl lactate, cetyl lactate, diethylene glycol monoethyl ether, and methyl laurate.

15. The composition of claim 1, wherein the composition comprises an emollient.

16. The composition of claim 15, wherein the emollient is the ester of a fatty acid.

17. The composition of claim 16, wherein the ester of a fatty acid is a triglyceride.

18. The composition of claim 17, wherein the triglyceride is caprylic/capric triglyceride.

19. The composition of claim 1, wherein the composition comprises a silicone fluid.

20. The composition of claim 19, wherein the silicone fluid comprises a trimethicone.

21. The composition of claim 20, wherein the trimethicone is phenyl trimethicone.

22. The composition of claim 1, wherein the composition further comprises an additional active.

23. The composition of claim 22, wherein the additional active is methyl salicylate, camphor, menthol, or a terpene.

24. The composition of claim 1, wherein the composition comprises a plurality of cannabinoids.

25. The composition of claim 1, wherein the cannabinoid is a naturally occurring cannabinoid.

26. The composition of claim 1, wherein the cannabinoid has been obtained by extraction from a naturally occurring source.

27. The composition of claim 1, wherein the cannabinoid is synthetic.

28. The composition of claim 1, wherein the cannabinoid is selected from cannabigerol (CBG), tetrahydrocannabinol (THC), cannabidiol (CBN), cannabichromene (CBC), cannabigerivarin (CBGV), tetrahydrocannabivarian (THCV), cannabidivarin (CBDV), and cannabichromevarin (CBCV).
